(19) United States
(12) Patent Application Publication
(10) Pub. No.: US 2006/0121009 A1
(43) Pub. Date: Jun. 8, 2006

(54) Apparatus and method for machining michrochamber for cell culture

(76) Inventor: Peter Bromley, Chene-Bougeries (CH)

Correspondence Address: GARY C. COHN, PLLC, 1147 NORTH FOURTH STREET, UNIT 6E, PHILADELPHIA, PA, 19123, US

(21) Appl. No.: 10/559,624

(22) PCT Filed: Jun. 16, 2004
(86) PCT No.: PCT/CH04/00363

§ 371 Date: Dec. 6, 2005

(30) Foreign Application Priority Data
Jun. 19, 2003 (EP) CHO3/00398

(51) Int. Cl. A61K 48/00; C12N 5/08; C12N 15/87

(52) U.S. Cl. 424/93.21; 435/455; 435/370

(57) ABSTRACT

A method for producing competent gene products in human cells, said method comprising the following steps: Providing a DNA construct in which a gene encoding a protein of interest is operably linked to a modified heat-inducible promoter, Introducing said DNA construct into a human cell line, either by transformation or by transfection to form transformed or transfected host cell lines, Subjecting said transformed or trasnfected cell lines to a transient increase in temperature and permitting the translation to protein to occur after the temperature has been returned to normal growth temperatures of the said host cells, whereby the production of said protein of interest occurs

The present invention relates to an Optimized Protein Production System using a Stable and Competent Human Hepatocyte Cell Line

### BACKGROUND

Therapeutic proteins have been known in the scientific and medical communities since the early twentieth century, but the small amounts harvestable from tissues and urine made therapeutic replacement difficult if not impossible. In the 1980s, advances in genomic technology have directly facilitated the identification, isolation, and characterization of genes responsible for the production of a great number of potential therapeutic proteins (‘biotherapeutics’).

Recombinant DNA technology allows the large-scale manufacture and production of many therapeutic proteins. This approach may use either a prokaryotic or eukaryotic source of cells for propagation. The function and efficacy of any protein—and, by proxy, a therapeutic protein, depends mainly on the gene sequence; however, several post-translational modifications to the protein may also play a crucial role in the ability of the protein to function with maximum efficacy.

Post-translational modifications (PTMs) change the property of the side groups of the amino acids—the building blocks of proteins—such that they alter protein function. Collectively, these post-translational modifications contribute significantly to the final structure and function of the protein. Therefore, when therapeutic proteins are made for use in humans, it is thought to be important to have the human pattern of post-translational accompaniments on the protein.

### INDUSTRY EXAMPLE

The original production of EPO in prokaryotic cells failed because the post-translational patterns imparted by the bacterial host-cell to its transfected human gene product were neither correct nor sufficient to confer appropriate levels of clinical efficacy to the drug. Therefore, the decision to produce EPO in mammalian—but not human—cells was an important point in the evolution of the drug.

Documented Differences Between Native Vs Recombinant Therapeutic Proteins

Reports on the importance of correct PTMs with respect to biological activity/therapeutic efficacy of biotherapeutics abound, particularly in the view of the impending flood of new biotherapeutic molecules in clinical development. Below are some examples of reported studies:

The glycosylation (a major PIN) of recombinant glycoproteins can profoundly affect their biological activities, including circulatory clearance rate, and recombinant proteins that are correctly glycosylated have significantly longer serum half lives than incorrectly glycosylated structures (Chitlaru T, Kronman C, Zeevi M, Kam M, Harel A, Ordentlich A, Velan B, Shafferman A (1998). Modulation of circulatory residence of recombinant acetylcholinesterase through biochemical or genetic manipulation of sialylation levels. Biochem J 1998 336:647-658.).

Important Glossary Terms:

Native protein: a protein that is routinely synthesized by a given tissue, organ, cell in the natural physiological state, in absence of any manipulation or engineering.

Recombinant protein: a gene product (protein) that is obtained after genetically engineering of a cell or organism.

Misaizu T, Matsuki S, Strickland T W, Takeuchi M, Kobata A, Takasaki S (1995). Role of antennary structure of N-linked sugar chains in renal handling of recombinant human erythropoietin. Blood 86:4097-4104.) found that the nature and degree of glycosylation of recombinant human erythropoeitin (EPO) profoundly affected the in vivo activity. Incorrect glycosylation patterns enhanced the total body clearance rate more than three times and resulted in a much lower activity for stimulation of erythroid progenitor cells.

In many cases, there are significant differences in glycosylation between native and recombinant glycoproteins, between recombinant forms expressed in different cell lines, and between related glycoproteins from different organs. Landberg E, Pahlsson P, Krotkiewski H, Stromqvist M, Hansson L, Lundblad A (1997). Glycosylation of bile-salt-stimulated lipase from human milk: comparison of native and recombinant forms. Arch Biochem Biophys 344:94-102.) found differences in the glycosylation of native and recombinant forms of bile-salt-stimulated lipase (BSSL) from human milk. Native BSSL contained a high amount of A2F family N-glycans whereas recombinant forms expressed in CHO or mouse fibroblast cell lines had predominantly A2 family glycans.

Jacquinot P M, Leger D, Wieruszeski J M, Coddeville B, Montreuil J, Spik G (1994). Change in glycosylation of chicken transferrin glycans biosynthesized during embryogenesis and primary culture of embryo hepatocytes. Glycobiology 4:617-624.) studying the oligosaccharides of transferrins from chicken serum, chicken embryo serum and from the culture medium of chicken embryo hepatocytes in primary culture found each had distinct glycosylation patterns.

Tanigawara Y, Hori R, Okumura K, Tsuji J, Shimizu N, Noma S, Suzuki J, Livingston D J, Richards S M, Keyes L D, et al. (1990). Pharmacokinetics in chimpanzees of recombinant human tissue-type plasminogen activator produced in mouse C127 and Chinese hamster ovary cells. Chem Pharm Bull (Tokyo) 1990 February; 38(2):517-22) demonstrated that two preparations of r-tPA's (recombinant tissue plasminogen activators) with different carbohydrate structures showed different pharmacokinetics, strongly suggesting that the carbohydrate structure can affect the biological activity, hence the therapeutic efficiency, of t-PA.

Why Native Proteins?

Recombinant proteins that lack correct human post-translational modifications can elicit neutralizing antibodies, resulting in reduced efficacy. Moreover, recombinantly-produced proteins are often cleared from circulation quickly, requiring frequent injections or pegylation to extend the half-life. “Pegylated” proteins are costly to produce and may lose some of their bioactivity, requiring higher dosage for the same efficacy.

Conversely, native proteins from human tissues are fully human glycosylated, providing products with clear activity/efficacy/safety advantages over current therapies:

Better and broader efficacy due to the presence of naturally-occurring glycan structures and subtypes on the final protein product as well as other post-translational modifications, more closely matching human therapeutic requirements;

Potentially fewer side effects due to lower dosage;

Longer half life in circulation and little allergic response due to proper PTMs;

Lower manufacturing cost due to the extraction of multiple proteins from a single cell source and manufacturing process;

Lower regulatory barrier due to use of a single immortalized human cell line and single manufacturing process;

Less frequent injections due to longer half-life of glycosylated proteins compared to non-glycosylated recombinant proteins produced in bacteria

Industry example: Glycogen storage disease type II (GSDII) is an autosomal recessive disorder caused by the deficiency of the protein GAA (acid alpha-glucosidase), a glycogen-degrading lysosomal enzyme.

This deficiency results in generalized deposition of lysosomal glycogen in almost all tissues of the body and can ultimately lead to cardiac failure before the age of two years (hence GSDII is a life-threatening condition).

Current treatment for the disease includes repairing the deficiency by injecting recombinant protein into the patient, made from either cultured Chinese Hamster Ovary (CHO) cells or secreted in the milk from rabbits that bear the transgene for the protein under a milk-specific promoter.

Both recombinant proteins produced are extremely inefficient in their uptake into and function in targeted tissues.

The NIH (US-National Institute of Health) announces a new technology that relates to the use of hepatocytes whether in culture or in vivo for the production of native human GAA.

The NIH approach is to use human hepatocytes to produce appropriate post-translational modification of the enzyme in cells by proper glycosylation, thereby producing a superior enzyme capable of being easily taken up and localized intracellularly in the target tissue. Once there, the enzyme digests glycogen present in lysosomes.

Proteins Naturally Produced by the Liver

The liver is one of the most promising organs/tissues to provide producer cells with a large spectrum potential for delivering native proteins with therapeutic interest, either as direct biological drugs or as validated drug targets for small drug molecule development Indeed, this organ synthesizes a host of important proteins, including enzymes, hormones, clotting factors, and immune factors. Several proteins synthesized by the liver are necessary for proper blood functioning; these include binding proteins and albumin, which helps maintain proper blood volume. Clotting factors produced by the liver include fibrinogen, prothrombin Factor II), Factors VII, VIII, IX, X and von Willebrand Factor. Acute phase proteins (APP) are another set of plasma proteins synthesized by the liver in response to tissue damage and inflammation associated with traumatic and/or infectious disease. Transferrin (Tf), alpha-2-macroglobulin (a2M), hemopexin are just some important acute proteins.

(Please refer to the APPENDIX for a comprehensive list of proteins produced by hepatocytes).

[MF notes: in case this issue is considered a valuable avenue to explore, all potential disease/therapeutic targets for each protein from the appendix list can be supplied]

Limitations of the Native Liver Proteins Approach

Primary hepatocytes do not proliferate, thus production of proteins from this type of cells requires a steady supply of new cellular preparations from human liver biopsies. This would represent a clumsy and expensive approach with many associated problems (QA, batch-to-batch variability, almost no standardization, regulatory hassle). This issue has been solved through TGE-Corp's in-licensing strategy (Multicell Technologies' unique immortalized and standardized fully competent human hepatocytes).

The remaining limit for producing native hepatic proteins for therapeutic or other uses is obviously determined by the set of proteins available at a decent yield from hepatic cells in culture. The APPENDIX section lists all major proteins that could potentially be manufactured under the label of ‘Native Proteins’.

Any other therapeutic protein candidate will have to be produced using genetic engineering strategies. However, even in this case, the human nature/origin of these cellular substrates (MCT's hepatic cell lines) should guarantee the best post-translational modification process currently available, thus leading to recombinant end-products with clear competitive advantages, including a favourable regulatory outlook.

Appendix 1

Basis for the Construction of High Efficiency Inducible Promoter—Gene Constructs, Expressing Therapeutic Proteins.

An expression vector will be constructed which allows for the selection of stable transfectants by selection for the zeocin antibiotic (Cayla) in both prokaryotic and eukaryotic cells. The Zeocin resistance gene will be obtained as a restriction digest fragment from the pZeoSV plasmid (Invitrogen) and will be ligated to a fragment containing a bacterial origin of replication obtained by PCR amplification from pUC19 (New England Biolabs). This ligation mixture will then be used to transform competent E. coli cells and the presence of the desired recombinant plasmid (pUC-Zeo) will be selected for on Zeocin-containing bacterial plates. A synthetic poly(A) sequence will be obtained as a restriction fragment from a digest of pGL3-Basic (Promega) and will be ligated into pUC-Zeo upstream of the HSP70B promoter and the desired recombinant (pUC-ZeoA) will selected for Zeocin resistance. The HSP70B driven expression cassette (Hi-Hot) will be obtained as a restriction fragment from the PCR amplification of pHi&Hot-MCS (V3) ()avid Harris, University of Arizona) from which an XhoI fragment in the multiple cloning region has been deleted. The Hi-Hot expression cassette will be ligated into pUC-ZeoA downstream of the synthetic poly(A) sequence and the desired recombinant (pHiHot-Zeo) will be selected for Zeocin resistance. Genes to be expressed under the control of the Hi-Hot system can be inserted into the unique XhoI and XbaI sites derived from the multiple cloning sequence of pHi&Hot-MCS. The Hi-Hot plasmid constructs are derived from those in Tsang et al., Biotechniques, 20:51-52, 1996 and Tsang et al., Biotechniques, 22:68.

In a comparison of expression systems in transient transfection experiments using the Lewis Lung Carcinoma cell line expressing Interleukin-2, the following results were obtained:

Hsp70B promoter—468 pg/ml of interleukin-2

CMV promoter—573 pg/ml of interleukin-2

Hi-Hot promoter—18,409 pg/ml of interleukin-2

The Hi-Hot Promoter was 39.3 Times Stronger Than the HSP70B Promoter and 32.1 Times Stronger Than the CMV Promoter

APPENDIX 2
SHORTLIST OF NATIVE HEPATIC PROTEINS

| Abbreviated | Protein Name(s) |
|---|---|
| 202 | Ifi 202 |
| 3alpha | 3alpha hydroxysteroid dihydrodiol dehydrogenase, AKR1C9, aldo-keto reductase |
| HSD/DD | 1C9 |
| A2-Mag | alpha2-macroglobulin |
| A2UG | alpha-2u globulin clone RAO 01 |
| A2UG207n | alpha-2u globulin 207 |
| AAT | alpha1-antitrypsin |
| ACC | acetyl-CoA carboxylase |
| ACE | angiotensin converting enzyme, DCP, DCP1, dipeptidyl carboxypeptidase 1, |
| | angiotensin I converting enzyme, peptidyl-dipeptidase A 1, CD143, dipeptydyl |
| | peptidase, Kininase II, ACE1 |
| ACO | acyl CoA oxidase |
| ACS | acyl-coenzyme A synthetase, long-chain-acyl-CoA synthetase |
| ADB | aldolase B |
| ADH1 | alcohol dehydrogenase 1, class I, alcohol dehydrogenase alpha subunit |
| ADH2 | alcohol dehydrogenase 2, class I |
| ADH3 | alcohol dehydrogenase 3, class I |
| AFP | alpha-fetoprotein |
| aG | alpha1-globin |
| AGG | Agamma globin, hemoglobin gamma chain |
| AGP | alpha-1-acid glycoprotein |
| ALAD | delta-aminolevulinate dehydratase, porphobilinogen synthase |
| ALB | albumin |
| ALDH-3 | Class 3 aldehyde dehydrogenase, fatty aldehyde dehydrogenase, aldehyde |
| | dehydrogenase microsomal |
| alpha1I3 | alpha1-inhibitor III |
| alpha2MR/LRP | alpha2-macroglobulin receptor/lipoprotein receptor-related protein |
| AML1 | acute myeloid leukemia 1, CBFA2, core-binding factor, runt domain, alpha subunit 2 |
| ANTG | angiotensinogen |
| AOX | acyl CoA oxidase, acyl-CoA: oxygen 2-oxidoreductase |
| APO(A) | apolipoprotein(a) |
| apoAI | apolipoprotein AI |
| ApoA-I | apolipoprotein A-I |
| apoAII | apolipoprotein AII |
| ApoA-IV | apolipoprotein A-IV |
| apoB | apolipoprotein B |
| apoC-III | apolipoprotein C-III |
| ApoD | apolipoprotein D |
| apoE | apolipoprotein E |
| apoVLDLII | very low density lipoprotein II, apovitellenin I |
| ARG | arginase |
| AT1a-R | angiotensin II type 1A receptor, angiotensin receptor (AT1) |
| ATP1A1 | Na, K-ATPase alpha 1 subunit, NAKA alpha 1 |
| beta2-AR | beta2-adrenergic receptor |
| beta-IFN | virus induced interferon-beta, IFN beta |
| Bf | complement factor B |
| BFIBR | fibrinogen B-beta subunit |
| BGP | biliary glycoprotein, C-CAM-1, cell CAM-1, BGP-1, BGP1, antigen CD66, |
| | CD66A antigen |
| C/EBPalpha | CCAAT/enhancer binding protein alpha |
| C3 | complement C3 |
| C4BP | C4b-binding protein |
| cAspAT | cytosolic aspartate aminotransferase |
| catalase | catalase, CAT, CAS1 |
| Cdc 7 | Cell division cycle 7 |
| CEBPA | CCAAT/enhancer binding protein alpha |
| CETP | cholesteryl ester transfer protein |
| COLIA1 | collagen, type I, alpha 1 |
| CRBPII | cellular retinol-binding protein II |
| CRP | C-reactive protein |
| CSP 1 | carbamoylphosphate synthetase 1 |
| Cyp11A | cytochrome P450 cholesterol side chain cleavage, P450scc |
| Cyp17 | cytochrome P-450 17alpha hydroxylase/C17-20 lyase, P450c17 |
| Cyp19 | aromatase, cytochrome P450XIXA1, estrogen synthetase, P450arom |
| CYP1A1 | cytochrome P450 family I A 1, P1-450, cytochrome P-450IA1 |
| Cyp21A1 | cytochrome P450 steroid 21-hydroxylase, P450C21, CypXXIA1, 21-OHase |
| CYP27 | Cytochrome P-450c27 |
| CYP2B2 | cytochrome P450IIB2 |
| CYP2C12 | Cytochrome P-450 family 2 C 12 |
| CYP7 | cholesterol 7-alpha-hydroxylase, cholesterol 7-alpha-monooxygenase, cytochrome |
| | P450 VII |
| DRA | MHC class II HLA-DRA |
| ei24 | etoposide induced protein 2.4, PIG8 |
| EKLF | erythroid Kruppel-like factor |
| Epo | erythropoietin |
| F10 | coagulation factor X, coagulation factor 10, stuart factor, f10 |
| F7 | factor VII, vitamin K-dependent coagulation protein |
| F8 | coagulation factor VIII, F8C, procoagulant component, antihemophilic factor, AHF |
| F9 | coagulation factor IX, Factor IX, Factor 9, christmas factor, FIX, f9 |
| FAS | fatty acid synthase |
| FASL receptor | Fas ligand receptor, Fas antigen, Fas death receptor, APO-1 antigen, CD95 antigen, |
| | TNFRSF6, APT1, FAS |
| FCH | ferrochelatase, protoheme ferro-lyase, heme synthetase |
| FGG | FIBRINOGEN GAMMA, FIBG |
| fibrinogen | gamma-fibrinogen subnit |
| gamma | |
| FPPS | farnesyl diphosphate synthase, geranyl-diphosphate: isopentenyl-diphosphate |
| | geranyltranstransferase |
| FTH | ferritin, heavy chain |
| G6Pase | glucose-6-phosphatase |
| GAD1 | glutamic acid decarboxylase 2, 67 kDa glutamate decarboxylase, GAD67 |
| GATA-1 | GATA-1 |
| Gbp-1 | guanylate-binding protein-1, mGBP-1, mag-1, macrophage activation-1 |
| Gbp-2 | guanylate-binding protein-2, mGBP-2, macrophage activator |
| GGG | Ggamma-globin |
| GK | glucokniase, hexokinase D, hexokinase type IV, HK4, ATP:D-hexose 6- |
| | phosphotransferase |
| GLOB-A | alpha1-globin, hemoglobin alpha1 chain |
| GLOB-B | beta-globin, hemoglobin beta chain |
| GLOB-BM | betaM-globin, hemoglobin beta major chain |
| GLOB-Z | zeta2-globin, hemoglobin z2 chain |
| GPA | glycophorin A |
| GPAT | mitochondrial glycerol-3-phosphate acyltransferase |
| GPC | glycophorin C |
| gpD | glycoprotein D, major subunit of Duffy blood group |
| GSHPx | cytosolic glutathione peroxidase, cellular glutathione peroxidase, GPX1 |
| gstA2 | Glutathione S-transferase A2 |
| GST-P | glutathione transferase P, placental glutathione transferase, glutation S-alkyl |
| | transferase |
| GSTYA | glutathione S-transferase Ya subunit |
| GST-Ya | glutathione S-transferase Ya subunit |
| H-2Dd | MHC class I H-2Dd |
| H-2Kb | MHC class I H-2Kb |
| H-2Kk | MHC class I H-2Kk |
| H-2Ld | MHC class I H-2Ld |
| HCEH | hepatic neutral cholesterol ester hydrolase, neutral cholesterol ester hydrolase, |
| | hepatic cholesterol ester hydrolase |
| HD | hydratase-dehydrogenase, enoyl-CoA hydratase/3-hydroxyacyl-CoA |
| | dehydrogenase, bifunctional enzyme |
| HG | haptoglobin |
| HK3 | hexokinase 3, HK III |
| HMGCR | HMG-CoA reductase, 3-hydroxy-3-methylglutaryl-coenzyme A reductase, 3- |
| | hydroxy-3-methylglutaryl-coA reductase |
| HMGCS | 3-hydroxy-3-methylglutaryl coenzyme A synthase, hydroxymethylglutaryl-CoA |
| | synthase, cytoplasmic, HMG-CoA synthase |
| HMGR | 3-hydroxy-3-methylglutaryl coenzyme A reductase |
| HNF-1 | hepatocyte nuclear factor-1, LFB1, LFB1/HNF1, HNF-1alpha |
| HNF-1beta | hepatocyte nuclear factor-1 beta, vHNF-1 |
| Hnf-3alpha | hepatocyte nuclear factor 3alpha |
| HNF3beta | hepatocyte nuclear factor 3 beta |
| HNF3gamma | hepatocyte nucler factor 3 gamma |
| HNF-4 | hepatocyte nuclear factor 4 |
| HO-1 | heme oxygenase 1, hsp32 |
| HPG | haptoglobin |
| Hpx | hemopexin |
| hsc70 | 72 kd heat shock cognate |
| hsc77 | heat shock cognate 73, HSC72, P72, HSC70, peroxisome proliferator binding |
| | protein |
| HSF1 | heat shock factor 1, HSTF1 |
| HSF2 | heat shock factor 2, HSTF2 |
| HSP105 | heat shock protein 105 |
| hsp25 | heat shock protein 25 |
| hsp84 | 84 kDa heat shock protein, HSP90B |
| HSPG2 | Basement membrane-specific heparan sulfate proteoglycan core protein, perlecan, |
| | HSPG, PLC |
| HTGL | hepatic triglyceride lipase, triacylglycerol lipase |
| ICAM-1 | intercellular adhesion molecule-1, major group rhinovirus receptor, HRV, CD54 |
| IFN-alpha | interferon-alpha |
| IGFBP-1 | insulin-like growth factor binding protein-1 |
| IFG-II | insulin-like growth factor II |
| iNOS | inducible nitric oxide synthase, NOS2, hiNOS, inducible NOS, macrophage-type |
| | NOS, iNOS, NOS, type II, NOS2A, hepatocyte OS, hep-NOS |
| IP-10 | inflammatory protein 10-kDa, Crg-2, gamma-IP10, C7, INP10, IFI10, SCYB10 |
| IR | insulin receptor |
| IRF-1 | interferon regulatory factor 1 |
| Itgb5 | integrin beta 5 |
| LPL | lipoprotein lipase |
| MCAD | medium-chain acyl-CoA dehydrogenase, acyl-CoA dehydrogenase, medium-chain |
| | specific, 2,3-oxidoreductase, ACADM |
| MDR1 | multidrug resistance, PGY1, P-glycoprotein |
| mdr1b | multidrug resistance 1b, P-glycoprotein, mdr1 |
| ME | malic enzyme, malate oxidoreductase |
| MHMGCS | mitochondrial 3-hydroxy-3-methylglutaryl coenzyme A synthase, mitochondrial |
| | HMG-CoA synthase |
| MT-I | metallothionein-I |
| MTP | microsomal triglyceride transfer protein |
| MXI1 | Max Interactor 1 |
| NFE2 | nuclear factor erythroid 2, nuclear factor (erythroid-derived 2), 45 kD, P45 NF-E2, |
| | leucine zipper protein NF-E2, NFE2, p45, NF-E2 |
| NPY | neuropeptide Y |
| NQO1 | NAD(P)H: quinone oxidoreductase, DT diaphorase, NMO1, NMOR, QR, QAO |
| OAS | 2′-5′ oligoadenylate synthetase, 2′-5′ A synthetase |
| ODC | ornithine decarboxylase, L-ornithine carboxilase |
| OTC | ornithine transcarbamylase, L-ornithine carbamoyltransferase |
| p44 | hepatitis-C-associated microtubular aggregate protein |
| P450c | cytochrome P450 family I A 1 |
| p53 | p53 tumor suppressor |
| PAH | Phenylalanine hydroxylase |
| PBGD | porphobilinogen deaminase, porphobilinogen ammonia-lyase (polymerizing), |
| | hydroxymethylbilane synthase |
| PCNA | proliferating cell nucleolar antigen p120 |
| PEPCK | phosphoenolpyruvate carboxykinase cytosolic, phosphopyruvate carboxylase |
| PFKFB | 6-phosphofructo-2-kinase/fructose-2,6-biphosphatase, 6PF-2-K/Fru-2,6-P2ASE, |
| | PFK-2/FBPase-2, RH2K |
| PGK-1 | phosphoglycerate kinase 1 somatic-type |
| pIgR | polymeric immunoglobulin receptor, polyimmunoglobulin receptor, polymeric IgA |
| | receptor |
| pk L | L-type pyruvate kinase |
| PPAR gamma | peroxisome proliferator activated receptor gamma, PPAR gamma 3, PPAR gamma 1 |
| PRLR | prolactin receptor |
| Pt | prothrombin, coagulation factor II, F2 |
| PYKM | M-type pyruvate kinase, PKM2, Pyruvate kinase, M |
| RAR-beta | retinoic acid receptor beta, Retinoic Acid Receptor beta 2, RAR-beta |
| RBTN2 | Rhombotin 2, T-cell translocation 2, TTG-2 |
| S14 | S14 product |
| SAA | serum amyloid A |
| SAA1 | serum amyloid A1 |
| SAA3 | serum amyloid A3 |
| SAAg9 | serum amyloid Ag9 |
| sgk | Serin/Threonine protein kinase |
| SIRP | signal-regulatory protein |
| SOD1 | Cu/Zn superoxide dismutase |
| Spi 2.1 | serine protease inhibitor 2.1, serpin |
| Spi 2.3 | serine protease inhibitor 2.3, SPI, serpin, contrapsin-like protease inhibitor |
| | precursor, kallikrein-binding protein, GHR-p63 |
| SPTB | beta-spectrin, SPTB1 |
| SREBP-2 | sterol regulatory element-binding protein-2 |
| SS | squalene synthase, farnesyl-diphosphate: farnesyl-diphosphate farnesyltransferase |
| Stat6 | Interleukin-4-induced transcription factor Stat6, IL-4 Stat |
| TAL1, | SCL, TCL5 |
| TAT | tyrosine aminotransferase |
| TF | transferrin |
| THPO | thrombopoietin, c-Mpl ligand, megakaryocyte colony stimulating factor, |
| | megakaryocyte growth and development factor |
| TO | tryptophan oxygenase |
| TOP3 | DNA topoisomerase III alpha, TOP3A, hTOP3 |
| TTR | transthyretin, prealbumin |
| VIT-A2 | vitellogenin A2 |
| VIT-B1 | vitellogenin B1 |
| VIT-II | vitellogenin II |
| vWF | von Willebrand factor |
| XOR | xanthine oxidoreductase, XDH, XDHA |
| zg | zeta-globin, hemoglobin z-chain |

Appendix 3
Existing Human Cell Line Production Systems Today

The initial success of biotechnology has been driven some 30 years ago, by the ability to transfer DNA sequences into living cells and make them produce therapeutic proteins/peptides on a large scale. The production of recombinant insulin was the first example of such a process, achieved in the 1970ies by introducing the insulin gene into bacteria, the simplest cell type available. These ‘engineered bacteria’ then produced insulin which to date is still successfully used to combat diabetes.

As the biotechnology industry became interested in the production of more complex protein drugs, more sophisticated producer cell types were required as a production platform. In the early 80ies, the large-scale manufacturing of therapeutic proteins in mammalian cells represented a major breakthrough. To date, animal cells, such as rodent cell lines, and in particular the Chinese Hamster Ovary cell line (CHO), represent the most important platform for the production of biopharmaceuticals, including some blockbuster biotech drugs.

However, in recent past clinical trials, in particular with recombinant murine antibodies, it rapidly became clear that nonhuman antibodies have the potential to elicit an immune response, thus blocking the efficacy of the treatment. These observations underscored the now recognized importance of correct ‘human-type’ post-translational modifications of complex protein products.

PER.C6™

The company Introgen b.v. (Leiden, NL), now Crucell, took the bioproduction process one step further from rodent to a human production platform. PER.C6™ is an expression platform that consists of a human cell line that can produce biopharmaceuticals for human therapeutic use. The PER.C6 cell line was generated from human retina-derived primary cells, which were immortalized by insertion of the adenovirus E1 gene. The cell line is derived from a single source of healthy human cells in a controlled and fully documented manner. The company has immortalized the cell so that it can replicate itself indefinitely, unlike normal human cells, a prerequisite feature essential to the production of recombinant biopharmaceutical products in sufficient quantities for commercial distribution.

Currently, Crucell promotes its PER.C6 production platform for the:

Production of monoclonal antibodies through rDNA technologies;

Production of various therapeutic proteins other than mAb's;

Vaccine production

Adenoviral vector production for gene therapy applications

Functional genomics

WHY PER.C6™

Crucell, the PER.C6 owner company, claims its platform to represent today's industry standard for applications such as 1-4 above. The main reason to this stems from the following assertion: “Crucell's Technology Maintains ‘Human’ Glycosylation Patterns.”

Major advantages of using PER.C6 cells for biopharmaceutical production:

Post-translational modifications (in particular Glycosylation): Optimal recombinant therapeutic protein products, in terms of half-life, biological activity and immunocompatibility contain human glycan structures (i.e. glycosylation patterns). Mammalian cells like CHO (the biotech ‘workhorse’) or other established non-human animal cell lines add non-human glycan structures to recombinant proteins or antibodies. PER.C6 cells perform human glycosylation patterns, resulting in higher biological activity and longer half-life.

Regulatory issues: As PER.C6 has been developed as a manufacturing platform for biopharmaceuticals, extensive documentation concerning the generation and characterization of the cell line has been assembled from the start. This documentation has been deposited as a biologics master file (BMF) with the FDA. PER.C6 has been approved for the generation of recombinant adenovirus for gene therapy trials, and has been accepted for Phase I/II clinical trials of an HIV vaccine being administered to both healthy and immunocompromised individuals.

Production yields—monoclonals: Currently used expression platforms for the production of monoclonal antibodies are CHO and NS/0 cells, with average expression yields amounting to approximately 0.5 g/l in final production processes. PER.C6 produce similar levels of antibodies in a non-optimized system and is expected to produce significantly more in optimized fed batch culture systems.

Intellectual Property: Crucell, Leiden, NL, wholly owns the technology and know-how associated with PER.C6, which translates into a transparent patent situation, which is far from being the case for all other production platforms for biopharmaceuticals.

Flexibility of use: The cells grow readily as adherent or suspension cultures, in serum-free and animal-component-free culture systems and can be easily transferred from one medium or growth condition to another.

Scalability: The presence of the adenovirus E1 gene inhibits apoptosis of PER.C6 cells, resulting in high viabilities when grown in batch production cultures. PER.C6 cells are easily scalable—the cells are currently grown in 2,500 L reactors and further upscaling is in progress.

Stability: Transfection of PER.C6 cells with expression plasmids is efficient, as is subsequent generation of stable sub-clones. Importantly, high expression levels of recombinant proteins are observed in the absence of gene amplification, giving a considerable time advantage over the use of cell lines that require amplification for efficient protein expression.

In a May 2002 hearing (http://www.fdagov/ohrms/dockets/ac/01/briefing/3750b1_{—}01.htm), the FDA considered the potential risks in using two novel cell substrates, i.e. HEK293 cells and PER.C6 cells. These cell lines were developed by transforming human embryonic kidney cells (293) and human embryonic retinal cells (PER.C6) with the transforming early region 1 (E1) of adenovirus type 5 (Ad5). Since cell lines such as 293 and PER.C6 express the AdS E1 region, they are able to complement the growth of defective AdS vectors which have been “crippled” by deletion of E1. Defective AdS vectors have been engineered to express foreign genes such as those from human immunodeficiency virus (HIV), the causative agent of AIDS, and vectors of this type are thought to have significant potential for vaccine development because of their demonstrated ability to generate cell-mediated immune responses to HIV. However, a feature of regulatory importance associated with AdS-transformed cells is their capacity to form tumors in immunodeficient animals such as nude mice.

In considering potential risks associated with the use of so-called Designer Cell Substrates—i.e., neoplastic cells derived from normal cells transformed by defined viral or cellular oncogenes or by immortalizing cellular genes (e.g., telomerase)—OVRR/CBER is considering the approach outlined within the framework of a “defined-risks” assessment Lewis et al., “A defined-risks approach to the regulatory assessment of the use of neoplastic cells as substrates for viral vaccine manufacture”, In Evolving Scientific and Regulatory Perspectives on Cell Substrates for Vaccine Development. Brown, Lewis, Peden, Krause (eds.) Develop. Biol. Stand. This framework is intended to examine, and wherever possible, to quantify the potential risk of “transmitting” the tumorigenic components of the cell substrate used for vaccine production, and determine whether that “transmission” might pose a risk, particularly an oncogenic risk, to vaccinees. Factors that could influence the risk associated with the use of Designer Cell Substrates include (a) the known mechanism of cell transformation leading to the development of tumorigenic cells; (b) residual cell substrate DNA; and (c) the presence of adventitious agents, especially oncogenic viruses.

Crucell

Crucell discovers and develops fully human biopharmaceuticals that utilize the immune system to combat disease. Crucell's proprietary technology platforms, MAbstract™, AdVac™ and PER.C6™, enable the discovery, development and production of novel antigens, Antibodies and Vaccines. Crucell offers its technology to pharmaceutical and biotechnology partners, and utilizes them to create Crucell's own product pipeline.

PER.C6™ is a human cell manufacturing platform, which has become the industry standard for production of recombinant adenoviral vectors. PER.C6™ has also proven to be a superior platform for the production of antibodies and vaccines.

Crucell has 19 licensees for its PER.C6™ technology, including Novartis, Pfizer, GSK, Aventis, Genzyme and Schering.

PER.C6™ in Crucell's Press Releases

PER.C6™ is a human cell platform for the development and manufacturing of bio-pharmaceutical products such as antibodies, proteins and vaccines. The superior yields and scalability of PER.C6, as well as the extensive history and safety documentation render PER.C6 the safe, cost effective and large-volume manufacturing platform that the pharmaceutical industry requires. Having launched the use of PER.C6 as a vaccine platform through an exclusive licensing agreement with Merck & Co. for their HIV vaccine, Crucell aims to expand its PER.C6 business in the field of vaccines. The current agreement with Rhein Biotech endorses this strategy.

1. A method for producing competent gene products in human cells, said method comprising the following steps:
Providing a DNA construct in which a gene encoding a protein of interest is operably linked to a modified heat-inducible promoter,
Introducing said DNA construct into a human cell line, either by transformation or by transfection to form transformed or transfected host cell lines,
Subjecting said transformed or trasnfected cell lines to a transient increase in temperature and permitting the translation to protein to occur after the temperature has been returned to normal growth temperatures of the said host cells, whereby the production of said protein of interest occurs

2. The method of claim one in which the modified heat inducible promoter is the Hi-Hot promoter.

3. The method of claim one where the human cell line is a competent human hepatocyte cell line.

4. The method of claim 3 where the gene expressed encodes a therapeutic protein.

5. The method of claim 3 where the gene expressed includes therepeutic genes such as interferons, interleukins, blood clotting factors, insulins, growth hormone, urokinase, EPO, TPA, FSH, somatostatin, antibodies, DNAase, myoglobin, pro- and anti-angiogenesis factors and proteins of veterinary interest.

6. The method of claim 3 where the gene expressed is a natural liver protein.
